(19) United States
(12) Patent Application Publication
(10) Pub. No.: US 2004/0126824 A1
(43) Pub. Date: Jul. 1, 2004

(54) Ovarian cancer-specific promoter

(76) Inventors: Hsi-Chou Cedric Liu, Des Moines, IA (US); Patricia A. Singer, Indianola, IA (US); Charles J. Link JR., Ames, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/643,829

(22) Filed: Aug. 19, 2003

Related U.S. Application Data
(60) Provisional application No. 60/404,695, filed on Aug. 20, 2002.

(51) Int. Cl. G01N 33/574; C07H 21/04; C07K 14/575

(52) U.S. Cl. 435/7.23; 435/69.1; 435/320.1; 435/325; 530/399; 536/23.5

(57) ABSTRACT

Novel CA125/M17S2 promoter sequences are disclosed. These promoters can be used in transgenic protocols for tissue specific expression and expression constructs, vectors and host cells.

### CROSS-REFERENCE TO RELATED APPLICATIONS

This application claims priority to U.S. provisional Application Serial No. 60/404,695, filed Aug. 20, 2002, which is herein incorporated by reference in its entirety.

### FIELD OF THE INVENTION

The present invention relates to newly identified polynucleotides and their variants, as well as their production and uses. In particular, the invention relates to promoter polynucleotides from the CA125 also known as NBR1, 1A1.3B, or M17S2 gene, herein referred to as CA125/M17S2 gene, as well as their variants and uses of the same in the treatment of disease and recombinant nucleotide techniques.

This application refers to several scientific or patent publications to describe the state of the art to which the invention pertains. Each of these publications is incorporated by reference herein, in its entirety.

### BACKGROUND OF THE INVENTION

Ovarian carcinoma is the most common cause of death from a gynecological malignancy in the United States. In the United States, a woman's lifetime risk of developing ovarian cancer is 1 in 70. Over two-thirds of the patients have advanced stage disease at presentation for which systemic chemotherapy is indicated after surgical debulking. Standard therapy consists of cisplatin or carboplatin with paclitaxel, and excellent response rates are observed; however, recurrence is common, and the majority of patients still die of disease progression. Ovarian cancer has a fairly unique natural history in humans. Even patients with advanced stages of the disease often have their disease confined to their abdomen for extended periods of time. The disease often stays localized to the abdomen and presents great difficulty for the patient by obstruction of the intestines or ureters. This natural history makes the possibility of locally-targeted therapy realistic. As a result, intraperitoneal therapies have been developed for the local administration of chemotherapeutic agents into the peritoneal cavity.

CA125 has been used as a marker for diagnosis of ovarian cancers since Bast et al. described it as an antigen that is elevated in serum of >80% of epithelial ovarian cancer patients (Bast et al., J. Clin. Invest., 68(5):1331-1337 (1981)). The initial cutoff value, 35 U/ml, of the normal CA125 level was first determined by a radioimmunoassay (Klug et al., Cancer Res. 44(3):1048-1053 (1984)). However, a double determinant immunoassay, which shows less variation than the original CA125 assay, set the cutoff value between 30 and 60 U/ml (Bon et al., Am. J. Obstet. Gynecol., 174:107-114 (1996)). Higher expression of CA125 in ovarian cancers is probably due to the interaction between specific transcription factors and the cis-elements located in the promoter region of CA125 gene. In addition, the introns of this gene may also contain regulatory elements of CA125 expression.

Conflicting data generated by biochemical determination of the CA125 antigen suggested that it is a protein, a mucin, a carbohydrate, or a membrane-associated glycoprotein (Nastad et al., Int. J. Biol. Markers, 13(4):196-199 (1998)). By using rabbit polyclonal antibodies raised against CA125, Campbell et al. screened an OVCA432 lambda ZAP cDNA library and isolated a gene called 1A1.3B (Campbell et al., Hum. Mol. Genet., 3(4):589-594 (1994)). The gene was later renamed NBR1 (meaning ‘next to BRCA1’) when it was mapped to the human chromosome region 17q21.1, the region in which the BRCA1 gene is located. This gene has been known as CA125 ovarian specific antigen, despite not being the one encoding CA125, which has generated confusion in the literature. The latest official denomination for this gene according to LocusLink database is M17S2, membrane component, chromosome 17, surface marker 2. As disclosed herein, Applicants refer to this gene as CA125/M17S2 gene. The predicted molecular mass of the CA125/M17S2 gene product is 108-kDa, and the CA125/M17S2 cDNA contains no nucleotide sequence for any transmembrane domains. When CA125/M17S2 was expressed as an epitope-tagged construct in epithelial cells, it was localized predominantly to a perinuclear, cytoplasmic location (Whitehouse et al., Eur. J. Biochem., 269:538-545 (2002)) and not in the membrane where CA125 is frequently localized by immunohistochemistry. Recently, a mucin gene, MUC16, was cloned by two groups and is a strong candidate for CA125 ovarian cancer antigen. (Yin et al., J. Biol. Chem., 276(29):27371-27375 (2001) and O'Brien et al., Tumour Biol., 22(6):348-366 (2001)).

Molecular expression analysis of the CA125/M17S2 promoter presented initially by our group and later confirmed by Hamada et al. (Hamada et al, Cancer Research 2003, 63:2506-12), demonstrates that the promoter of the CA125/M17S2 gene has specific expression activity in ovarian cancer cells, whether they express the CA125 antigen or not, which makes CA125/M17S2 a better diagnostic marker and also a new therapeutic tool for gene therapy applications in ovarian cancers.

A major, indeed the overwhelming, obstacle to cancer therapy is the problem of selectivity; that is, the ability to inhibit the multiplication of tumor cells, while leaving unaffected the function of normal cells. Thus, the therapeutic ratio, or ratio of tumor cell killing to normal cell killing of traditional tumor chemotherapy, is only 1.5:1. Thus, more effective treatment methods for therapy and prophylaxis of ovarian cancer are needed.

The use of gene therapy in cancer treatment presents many of the same disadvantages as other therapeutic approaches, such as chemotherapy and radiation therapy. Problems with current state-of-the-art gene therapy strategies include the inability to deliver the therapeutic gene specifically to the target cells. This leads to toxicity in cells that are not the intended targets. For example, manipulation of the p53 gene suppresses the growth of both tumor cells and normal cells, and intravenous administration of tumor necrosis factor alpha (TNF-alpha) induces systemic toxicity with such clinical manifestations as fever and hypertension.

Attempts have been made to overcome these problems. These include such strategies as the use of tissue-specific receptors to direct the genes to the desired tissues (see, e.g., Kasahara et al., Science 266:1373-6 (1994)), the use of heat (see, e.g., Voellmy et al., Proc. Natl. Acad. Sci. USA 82:4949-53 (1985)) or ionizing radiation inducible enhancers and promoters (see, e.g., Trainman et al., Cell 46:567-74 (1986); Prowess et al., Proc. Natl. Acad. Sci. USA 85:7206-10 (1988)), and the use of tissue-specific promoters to limit gene expression to specific tissues to enhance expression of the therapeutic gene in a temporally and spatially controlled manner. A well-documented example of this latter approach involves the use of the prostate-specific antigen (PSA) promoter in recombinant constructs to direct expression of therapeutic genes to prostatic tissue. See, e.g., U.S. Pat. No. 5,648,478.

Given the deficiency in the prior art regarding the lack of effective means for inhibiting unwanted and toxic side effects of gene therapy treatments for cancer, and the discovery of the ability of the PSA promoter to direct expression of therapeutic genes to cancerous prostatic tissue, it would be desirable to obtain tissue-specific promoters that could direct the expression of therapeutic genes for the treatment of ovarian cancer. The present invention fulfills this longstanding need and desire in the art.

### BRIEF SUMMARY OF THE INVENTION

The present invention relates to CA125/M17S2, and in particular CA125/M17S2 promoter polynucleotides, recombinant materials and methods for their production. In another aspect, the invention relates to methods for using such polynucleotides, including transgenic protocols to provide for temporal and spatial expression of recombinant polynucleotides operably linked thereto.

According to the invention, an isolated nucleic acid molecule comprising a CA125/M17S2 promoter is provided. In a preferred embodiment, the isolated nucleic acid molecule comprises a human CA125/M17S2 promoter. In an even more preferred embodiment, the invention comprises the sequence of SEQ ID NO: 1, fragments thereof or its conservatively modified variants.

The invention also comprises nucleic acid constructs wherein the CA125/M17S2 promoter is operably linked to a gene of interest, replication and expression vectors incorporating these constructs, and host cells transformed with these vectors.

The specificity of the promoter can also be used in gene therapy protocols to direct expression of recombinant nucleotides in ovarian cancer cells. This includes expression constructs, vectors (replication and expression), and transformed recipient cells.

Other features and advantages of the present invention will be better understood by reference to the drawings, detailed descriptions and examples that follow.

### BRIEF DESCRIPTION OF THE DRAWINGS

FIG. 1 is the sequence of CA125p1431 DNA fragment amplified with PCR. This DNA fragment contains exon 1A and partial sequence of exon 1B of the CA125/M17S2 gene. The sequence of exon 1A is underlined, and the partial exon 1B sequence is in bold-type letters.

FIG. 2 shows the activity of CA125P1431, a CA125 promoter, in three different ovarian cancer cell lines, SK-OV-3, IGROV, and OVCAR3. CA125 is known to be expressed in OVCAR3, and is known not to be expressed in either SK-OV-3 or IGROV. As clearly indicated, CA125p1431 is more active in OVCAR3 cells than in SK-OV-3 or IGROV cells.

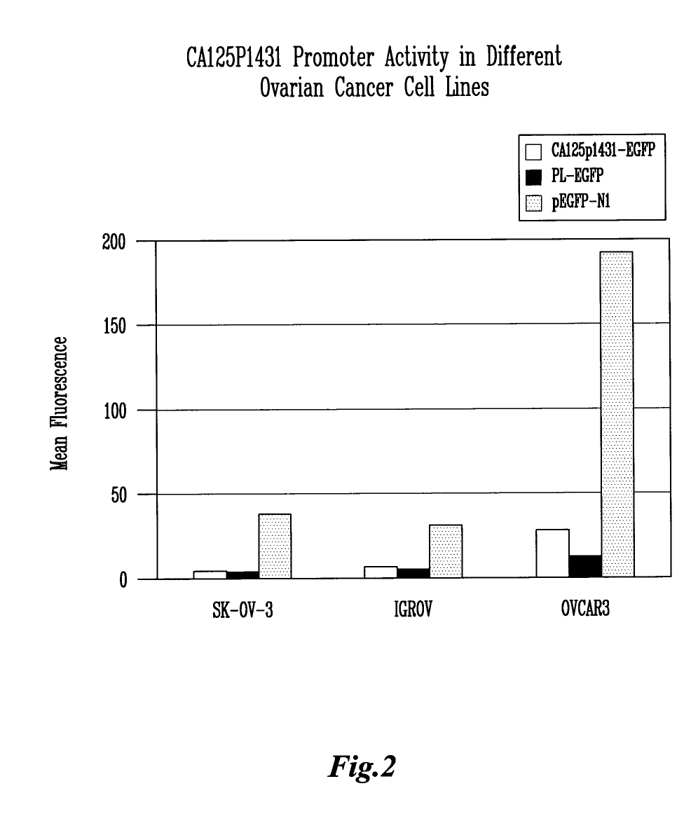

FIG. 3 is a schematic representation of alternatively spliced forms of human CA125/M17S2 mRNA. Human CA125/M17S2 gene is located on chromosome 17. The ubiquitous CA125/NBR1 transcript, mRNA variant 1, is expressed from exon 1B. Two alternative isoforms, mRNA variant 2 and mRNA variant 3, are transcribed from exon 1A, but the mRNA variant 3 contains a shorter sequence of exon 1A (shown as 1A′). Translational initiation codon ATG of the open reading frame of CA125/M17S2 protein is located in exon 2. 3′ UTR: untranslated region at the 3′ end of mRNA.

FIG. 4 is a schematic representation of plasmid DNAs used for analysis of promoter activities of a series of deletions of CA125p1431 fragment. The CA125p1431 and its deletions of various lengths (CA684, CA554, CA424, CA294, and CA164) were amplified by PCRs and then cloned into the restriction sites upstream of the enhanced green fluorescence protein (EGFP) open reading frame in pPL-EGFP, and pCA164-EGFP, respectively. The CA125p1431-containing sequence at the 5′ end of human CA125/M17S2 gene is shown on top as a reference. The numbers on top of CA125p1431 fragment are used to denote the nucleotide positions as shown in FIG. 1. A fragment containing the bovine growth hormone polyA signal (BGH polyA) was used to construct pPolyA-EGFP as a negative control plasmid.

FIG. 5 shows promoter activities of CA125p1431 deletions determined by EGFP expression. pCA1431-EGFP, pCA684-EGFP, pCA554-EGFP, pCA424-EGFP, pCA294-EGFP, pCA164-EGFP, and pPolyA-EGFP plasmid DNAs were individually transfected into each of the OVCAR3, IGROV, SK-OV-3, PA-1, SW626, HEK293, and DU145 cell lines to determine the promoter activities of CA125p1431 fragment and its serial deletions (CA684, CA554, CA424, CA294, and CA164). pPolyA-EGFP was used as a negative control plasmid. The promoter activities expressed as mean fluorescence values were determined by analyzing EGFP expression of transfected cells with fluorescence activated cell sorting (FACS).

### DETAILED DESCRIPTION OF THE INVENTION

I. Definitions

Various terms relating to the compositions and methods of the present invention are used herein above and also throughout the specification and claims.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in THE NEW IEEE STANDARD DICTIONARY OF ELECTRICAL AND ELECTRONICS TERMS, 5^{th} ed. (1993). The terms defined below are more fully defined by reference to the specification as a whole.

As used herein, “therapeutic gene” means DNA encoding an amino acid sequence corresponding to a functional protein capable of exerting a therapeutic effect on ovarian cancer cells or having a regulatory effect on the expression of a function in ovarian cells.

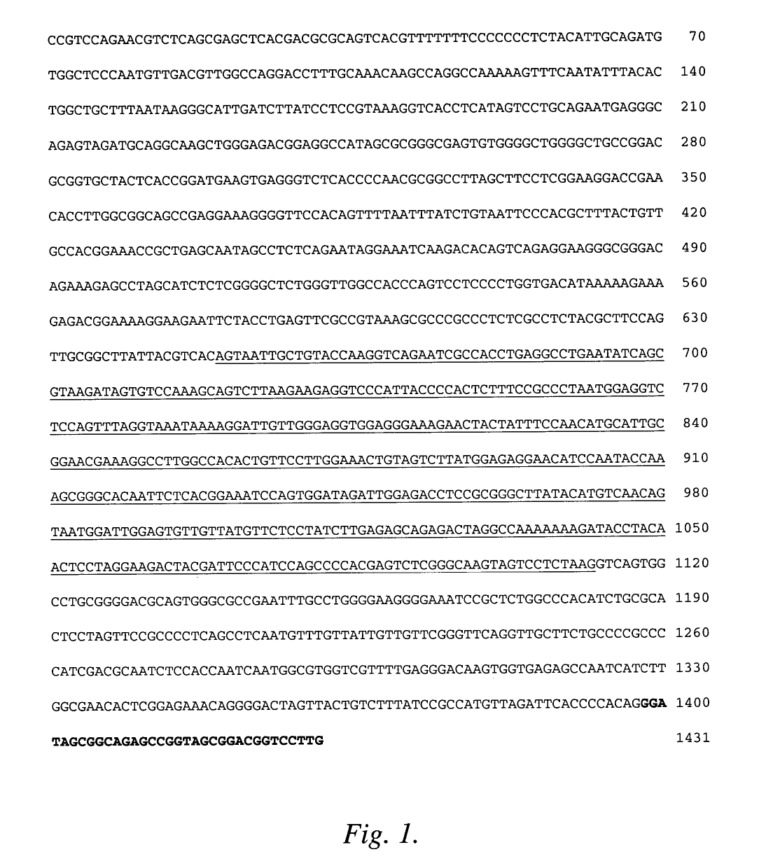

As used herein, “CA125/M17S2 promoter” is intended to refer to a sequence of DNA or a fragment thereof derived from within or upstream of the CA125/M17S2 gene (also known as 1A1-3B, IAI-3B, NBR1, or M17S2) that is capable of directing the expression of the CA125/M17S2 gene. As used herein, “CA125p1431” is intended to refer to a 1431-base pair fragment derived from the 5′ flanking region of the CA125/M17S2 gene that is capable of directing the expression of the CA125/M17S2 gene, as well as any genes which are operably attached thereto. The sequence of CA125p1431 is shown in FIG. 1, and is embodied in SEQ ID NO:1. As used herein, “CA1251M17S2 promoter activity” is that activity which results in the expression of genes that are operably linked to a CA125/M17S2 promoter in a spatial and temporal way that is similarly to the CA125 expression.

As used herein “enhancer element” is a base sequence that increases the rate of transcription of the therapeutic genes or genes of interest but does not have promoter activity. An enhancer can be moved upstream, downstream, and to the other side of the CA125 promoter without significant loss of activity.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., DIAGNOSTIC MOLECULAR MICROBIOLOGY: PRINCIPLES AND APPLICATIONS, Persing et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention. With respect to noncoding nucleic acid sequences conservatively modified, variants refer to these variants which retain the promoter activity of the sequence as determined by the assays disclosed herein.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, PROTEINS, Creighton, W.H. Freeman and Company (1984).

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed.

As used herein, “full-length sequence” in reference to a specified polynucleotide or its encoded protein means having the entire amino acid sequence of, a native (non-synthetic), endogenous, biologically active form of the specified protein. Methods to determine whether a sequence is full-length are well known in the art including such exemplary techniques as northern or western blots, primer extensions, S1 protection, and ribonuclease protection. See, e.g., PLANT MOLECULAR BIOLOGY: A LABORATORY MANUAL, Clark, ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

With respect to proteins or peptides, the terms “isolated protein (or peptide)” and “isolated and purified protein (or peptide)” are sometimes used herein. These terms may refer to a protein that has been sufficiently separated from other proteins with which it would naturally be associated, so as to exist in “substantially pure” form. Alternatively, these terms may refer to a protein produced by expression of an isolated nucleic acid molecule.

As used herein “nucleic acid molecule” includes both DNA and RNA and, unless otherwise specified, includes both double-stranded and single-stranded nucleic acids. Also included are hybrids such as DNA-RNA hybrids. Reference to a nucleic acid sequence can also include modified bases as long as the modification does not significantly interfere either with binding of a ligand such as a protein by the nucleic acid or Watson-Crick base pairing. A “nucleic acid molecule” can comprise two or more nucleotide sequences associated together.

With reference to nucleic acid molecules, the term “isolated nucleic acid molecule” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid molecule” may comprise a DNA molecule inserted into a vector, such as a plasmid or virus vector, or integrated into the genomic DNA of a prokaryote or eukaryote. An “isolated nucleic acid molecule” may also comprise a cDNA molecule.

With respect to RNA molecules, the term “isolated nucleic acid molecule” primarily refers to an RNA molecule encoded by an isolated DNA molecule as defined above. Alternatively, the term may refer to an RNA molecule that has been sufficiently separated from RNA molecules with which it would be associated in its natural state (i.e. in cells or tissues), such that it exists in a “substantially pure” form (the term “substantially pure” is defined below).

By “host cell” is meant a cell which contains a vector and supports the replication and/or expression of the vector. Host cells may be prokaryotic cells such as E. coli, or eukaryotic cells such as yeast, insect, amphibian, or mammalian cells.

The term “hybridization complex” includes reference to a duplex nucleic acid structure formed by two single-stranded nucleic acid sequences selectively hybridized with each other.

The term “introduced,” in the context of inserting a nucleic acid into a cell, means “transfection,” “transformation,” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

As used herein, “localized within the chromosomal region defined by and including,” with respect to particular markers, includes reference to a contiguous length of a chromosome delimited by and including the stated markers.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as METHODS IN ENZYMOLOGY, GUIDE TO MOLECULAR CLONING TECHNIQUES (vol. 152), Berger et al., eds., Academic Press, Inc., San Diego, Calif. (1987); MOLECULAR CLONING: A LABORATORY MANUAL, 3rd ed., Sambrook et al., Cold Spring Harbor Laboratory Press, Cold Spring Harbor, N.Y. (2001); and CURRENT PROTOCOLS IN MOLECULAR BIOLOGY, Ausubel et al., John Wiley & Sons (1999).

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide,” “peptide,” and “protein” are also inclusive of modifications including, but not limited to, phosphorylation, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention. With respect to a protein, the term “N-terminal region” shall include approximately 50 amino acids adjacent to the amino terminal end of a protein.

As used herein, “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

The terms “residue,” “amino acid residue,” and “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The terms “stringent conditions” and “stringent hybridization conditions” include reference to conditions under which a probe will hybridize to its target sequence, to a detectably greater degree than to other sequences (e.g., at least 2-fold over background). Stringent conditions are sequence-dependent and may be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth et al., Anal. Biochem., 138:267-84 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in LABORATORY TECHNIQUES IN BIOCHEMISTRY AND MOLECULAR BIOLOGY-HYBRIDIZATION WITH NUCLEIC ACID PROBES, Part I, Tijssen, ed., Elsevier Science Ltd. (1993). Unless otherwise stated, in the present application high stringency is defined as hybridization in 4×SSC, 5× Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C., two to three times for at least 15 minutes.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g., chromatographic methods, agarose or polyacrylamide gel electrophoresis, HPLC analysis, and the like).

Nucleic acid sequences and amino acid sequences can be compared using computer programs that align the similar sequences of the nucleic or amino acids thus define the differences. The BLAST programs (NCBI) and parameters used therein are used by many practitioners to align amino acid sequence fragments. However, equivalent alignments and similarity/identity assessments can be obtained through the use of any standard alignment software. For instance, the GCG Wisconsin Package version 9.1, available from the Genetics Computer Group in Madison, Wis., and the default parameters used (gap creation penalty=12, gap extension penalty=4) by Best-Fit program may also be used to compare sequence identity and similarity.

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor, J. Theor. Biol. 119:205 (1986). When referring to nucleic acid molecules, “percent identical” refers to the percent of the nucleotides of the subject nucleic acid sequence that have been matched to identical nucleotides by a sequence analysis program.

With respect to oligonucleotides or other single-stranded nucleic acid molecules, the term “specifically hybridizing” refers to the association between two single-stranded nucleic acid molecules of sufficiently complementary sequence to permit such hybridization under pre-determined conditions generally used in the art, i.e. conditions of stringency (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, when the sequence is expressed.

The terms “operably linked” and “operably inserted” mean that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g., enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter,” “promoter region,” and “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase. Thus as used herein the term promoter shall include any portion of genomic DNA disclosed herein which is capable of initiating expression of operably linked sequences at levels detectable above background.

A “vector” is a replicon, such as plasmid, phage, cosmid, or virus to which another nucleic acid segment may be operably inserted so as to bring about the replication or expression of the segment.

The terms “nucleic acid construct” and “DNA construct” are sometimes used to refer to a coding sequence or sequences operably linked to appropriate regulatory sequences and inserted into a vector for transforming a cell. These terms may be used interchangeably with the term “transforming DNA.” Such a nucleic acid construct may contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct,” as defined above, is also used to refer to a heterologous region, particularly one constructed for use in transformation of a cell. As used herein, “heterologous,” in reference to a nucleic acid, further refers to a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

II. DESCRIPTION

According to the invention, a CA125/M17S2 promoter has been discovered. The discovery of this promoter leads to several transgenic protocols which take advantage of the regulation of expression of therapeutic genes to aid in treatment of disease, particularly ovarian cancer.

The present invention provides an isolated nucleic acid molecule comprising a CA125/M17S2 promoter, e.g., the CA125/M17S2 promoter as set forth in SEQ ID NO:1 and FIG. 1, having a nucleic acid sequence beginning with cytosine at nucleotide position 1 and ending with guanine at nucleotide position 1431.

Preferably, the nucleic acid molecule further comprises a therapeutic gene.

In one embodiment, the isolated nucleic acid molecule of the invention combines the CA125/M17S2 promoter with an enhancer element. In a preferred embodiment, the enhancer element can be a portion of the CMV LTR or other enhancers, e.g., SV40 enhancer sequences, MMTV LTR. Other promoters are possible.

Preferably, the enhancer element, e.g., the CMV LTR, is positioned 5′ of the CA125/M17S2 promoter in the molecule.

The nucleic acid molecule of the invention may be modified, i.e., by sequence mutations, deletions, and insertions, so as to produce derivative molecules. Other modifications include deleting sequences that are nonfunctional in the CA125/M17S2 promoter, and adding enhancers, thereby improving the efficiency of the CA125/M17S2 promoters. Enhancers may function in a position-independent manner and can be within or downstream of the transcribed region.

Derivative molecules would retain the functional property of the CA125/M17S2 promoter, namely, the molecule having such substitutions will still permit the ovarian tissue specific expression of the gene of interest. Modification is permitted so long as the derivative molecules retain its increased potency compared to CA125/M17S2 promoter alone and its tissue specificity.

In a preferred embodiment, a nucleic acid construct and/or a vector can be constructed by inserting a heterologous polynucleotide (i.e., therapeutic gene) into the nucleic acid molecule of the invention downstream of the modified CA125/M17S2 promoter.

Examples of therapeutic genes include suicide genes. These are genes sequences the expression of which produces a protein or agent that inhibits ovarian tumor cell growth or ovarian tumor cell death. Suicide genes include genes encoding enzymes, oncogenes, tumor suppressor genes, genes encoding toxins, genes encoding cytokines, or a gene encoding oncostatin. The purpose of the therapeutic gene is to inhibit the growth of or kill ovarian cancer cells or produce cytokines or other cytotoxic agents which directly or indirectly inhibit the growth of or kill the ovarian cancer cells.

Suitable enzymes include thymidine kinase (TK), xanthine-guanine phosphoribosyltransferase (GPT) gene from E. coli or E. coli cytosine deaminase (CD), or hypoxanthine phosphoribosyl transferase (HPRT).

Suitable oncogenes and tumor suppressor genes include neu, epidermal growth factor (EGF), ras (including H, K, and N ras), p53, Retinoblastoma tumor suppressor gene (Rb), Wilm's Tumor Gene Product, Phosphotyrosine Phosphatase (PTPase), and nm23. Suitable toxins include Pseudomonas exotoxin A and S; diphtheria toxin (DT); E. coli LT toxins, Shiga toxin, Shiga-like toxins (SLT-1, -2), ricin, abrin, supporin, and gelonin.

Suitable cytokines include interferons, GM-CSF interleukins, tumor necrosis factor (TNF) (Wong et al., Science 228:810 (1985); WO9323034 (1993); Horisberger et al., J. Virol. 64(3):1171-81 (1990); Li et al., J. Immunol. 148(3):788-94 (1992); Pizarro et al., Transplantation 56(2):399-404 (1993); Breviario et al., J. Biol. Chem. 267(31):22190-7 (1992); Espinoza-Delgado et al., J. Immunol. 149(9):2961-8 (1992); Algate et al., Blood 83(9):2459-68 (1994); Cluitmans et al., Ann. Hematol. 68(6):293-8 (1994); Lagoo et al., J. Immunol. 152(4):1641-52 (1994); Martinez et al., Transplantation 55(5):1159-66 (1993); Pang et al., Clin. Exp. Immunol. 96(3):437-43 (1994); Ulich et al., J. Immunol. 146(7):2316-23 (1991); Mauviel et al., J. Immunol. 149(9):2969-76 (1992)).

Growth factors include Transforming Growth Factor (TGF)-alpha, TGF-beta, and cytokine colony stimulating factors (Shimane et al., Biochem. Biophys. Res. Comm. 199(1):26-32 (1994); Kay et al., J. Exp. Med. 173(3):775-8 (1991); de Wit et al., British J. Haematol. 86(2):259-64 (1994); Sprecher et al., Archives Virol. 126(1-4):253-69 (1992)).

Preferred vectors for use in the methods of the present invention are viral including adenoviruses, retroviral vectors, a herpes simplex vector, and adeno-associated viral (AAV) vectors.

The viral vector selected should meet the following criteria: 1) the vector must be able to infect the tumor cells and thus viral vectors having an appropriate host range must be selected; 2) the transferred gene should be capable of persisting and being expressed in a cell for an extended period of time; and 3) the vector should be safe to the host and cause minimal cell transformation. Retroviral vectors and adenoviruses offer an efficient, useful, and presently the best-characterized means of introducing and expressing foreign genes efficiently in mammalian cells. These vectors have very broad host and cell type ranges, express genes stably and efficiently. The safety of these vectors has been proved by many research groups. In fact, many are in clinical trials.

Other virus vectors that may be used for gene transfer into cells for correction of disorders include retroviruses such as Moloney murine leukemia virus (MoMuLV); papovaviruses such as JC, SV40, polyoma, adenoviruses; Epstein-Barr Virus (EBV); papilloma viruses, e.g., bovine papilloma virus type I (BPV); vaccinia and poliovirus and other human and animal viruses.

Adenoviruses have several properties that make them attractive as cloning vehicles (Bachettis et al., Proc. Natl. Acad. Sci. USA 74:1590 (1977); Berkner, Biotechniques 6:616 (1988); Ghosh-Choudhury et al., Gene 50:161 (1986); Hag-Ahmand et al., J. Virol. 57:257 (1986); Rosenfeld et al., Science 252:431 (1991)). For example, adenoviruses possess an intermediate sized genome that replicates in cellular nuclei; many serotypes are clinically innocuous; adenovirus genomes appear to be stable despite insertion of foreign genes; foreign genes appear to be maintained without loss or rearrangement; and adenoviruses can be used as high level transient expression vectors with an expression period up to 4 weeks to several months. Extensive biochemical and genetic studies suggest that it is possible to substitute up to 7-7.5 kb of heterologous sequences for native adenovirus sequences generating viable, conditional, helper-independent vectors (Kaufman, Proc. Natl. Acad. Sci. USA 82:689 (1985)).

AAV is a small human parvovirus with a single stranded DNA genome of approximately 5 kb. This virus can be propagated as an integrated provirus in several human cell types. AAV vectors have several advantages for human gene therapy. For example, they are trophic for human cells but can also infect other mammalian cells; (2) no disease has been associated with AAV in humans or other animals; (3) integrated AAV genomes appear stable in their host cells; (4) there is no evidence that integration of AAV alters expression of host genes or promoters or promotes their rearrangement; (5) introduce genes can be rescued from the host cell by infection with a helper virus such as adenovirus.

HSV-1 vector system facilitates introduction of virtually any gene into non-mitotic cells (Geller et al., Proc. Natl. Acad. Sci. USA 87:8950 (1990)).

Another vector for mammalian gene transfer is the bovine papilloma virus-based vector (Sarver et al., Mol. Cell Biol. 1:486 (1981)).

Vaccinia and other poxvirus-based vectors provide a mammalian gene transfer system. Vaccinia virus is a large double-stranded DNA virus of 120 kilodaltons (kd) genomic size (Panicali et al., Proc. Natl. Acad. Sci. USA 79:4927 (1982); Smith et al., Nature 302:490 (1983)).

Retroviruses are packages designed to insert viral genes into host cells (Guild et al., J. Virol. 62:795 (1988); Hock et al., Nature 320:275 (1986)). The basic retrovirus consists of two identical strands of RNA packaged in a proviral protein. The core is surrounded by a protective coat called the envelope, which is derived from the membrane of the previous host but modified with glycoproteins contributed by the virus.

Retroviral vectors are generally constructed such that the majority of sequences coding for the structural genes of the virus are deleted and replaced by the gene(s) of interest. Most often, the structural genes (i.e., gag, pol, and env) are removed from the retroviral backbone using genetic engineering techniques known in the art. This may include digestion with the appropriate restriction endonuclease or, in some instances, with Bal 31 exonuclease to generate fragments containing appropriate portions of the packaging signal.

Genes of interest can be incorporated into the proviral backbone in several general ways. The most straightforward constructions are ones in which the structural genes of the retrovirus are replaced by a single gene which then is transcribed under the control of the viral regulatory sequences within the long terminal repeat (LTR). In the present invention, the retroviral vector can comprise a gene of interest (i.e., therapeutic gene) under the control of a CA125 promoter. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Efforts have been directed at minimizing the viral component of the viral backbone, largely in an effort to reduce the chance for recombination between the vector and the packaging-defective helper virus within packaging cells. A packaging-defective helper virus is necessary to provide the structural genes of a retrovirus, which have been deleted from the vector itself.

In one embodiment, the retroviral vector may be one of a series of vectors described in Bender et al., J. Virol. 61:1639-49 (1987), based on the N2 vector (Armentano et al., J. Virol. 61:1647-50) containing a series of deletions and substitutions to reduce to an absolute minimum the homology between the vector and packaging systems. These changes have also reduced the likelihood that viral proteins would be expressed. In the first of these vectors, LNL-XHC, there was altered, by site-directed mutagenesis, the natural ATG start codon of gag to TAG, thereby eliminating unintended protein synthesis from that point.

In Moloney murine leukemia virus (MoMuLV), 5′ to the authentic gag start, an open reading frame exists which permits expression of another glycosylated protein (pPr80^{gag}). Moloney murine sarcoma virus (MoMuSV) has alterations in this 5′ region, including a frameshift and loss of glycosylation sites, which obviate potential expression of the amino terminus of pPr80^{gag}. Therefore, the vector LNL6 was made, which incorporated both the altered ATG of LNL-XHC and the 5′ portion of MoMuSV. The 5′ structure of the LN vector series thus eliminates the possibility of expression of retroviral reading frames, with the subsequent production of viral antigens in genetically transduced target cells. In a final alteration to reduce overlap with packaging-defective helper virus, Miller has eliminated extra env sequences immediately preceding the 3′ LTR in the LN vector (Miller et al., Biotechniques 7:980-90 (1989)).

The paramount need that must be satisfied by any gene transfer system for its application to gene therapy is safety. Safety is derived from the combination of vector genome structure together with the packaging system that is utilized for production of the infectious vector. Miller et al. have developed the combination of the pPAM3 plasmid (the packaging-defective helper genome) for expression of retroviral structural proteins together with the LN vector series to make a vector packaging system where the generation of recombinant wild-type retrovirus is reduced to a minimum through the elimination of nearly all sites of recombination between the vector genome and the packaging-defective helper genome (i.e., LN with pPAM3).

In one embodiment, the retroviral vector may be a Moloney Murine Leukemia Virus of the LN series of vectors, such as those hereinabove mentioned, and described further in Bender et al., J. Virol. 61:1639-49 (1987) and Miller et al., Biotechniques 7:980-90 (1989). Such vectors have a portion of the packaging signal derived from a mouse sarcoma virus, and a mutated gag initiation codon. The term “mutated” as used herein means that the gag initiation codon has been deleted or altered such that the gag protein or fragment or truncations thereof, are not expressed.

In another embodiment, the retroviral vector may include at least four cloning, or restriction enzyme recognition sites, wherein at least two of the sites have an average frequency of appearance in eukaryotic genes of less than once in 10,000 base pairs; i.e. the restriction product has an average DNA size of at least 10,000 base pairs. preferred cloning sites are selected from the group consisting of NotI, SnaBI, SalI, and XhoI. In a preferred embodiment, the retroviral vector includes each of these cloning sites.

When a retroviral vector including such cloning sites is employed, there may also be provided a shuttle cloning vector which includes at least two cloning sites which are compatible with at least two cloning sites selected from the group consisting of NotI, SnaBI, SalI, and XhoI located on the retroviral vector. The shuttle cloning vector also includes at least one desired gene which is capable of being transferred from the shuttle cloning vector to the retroviral vector.

The shuttle cloning vector may be constructed from a basic “backbone” vector or fragment to which are ligated one or more linkers which include cloning or restriction enzyme recognition sites. Included in the cloning sites are the compatible, or complementary cloning sites hereinabove described. Genes and/or promoters having ends corresponding to the restriction sites of the shuttle vector may be ligated into the shuttle vector through techniques known in the art.

The shuttle cloning vector can be employed to amplify DNA sequences in prokaryotic systems. The shuttle cloning vector may be prepared from plasmids generally used in prokaryotic systems and in particular in bacteria. Thus, for example, the shuttle cloning vector may be derived from plasmids such as pBR322; pUC 18; etc.

The vector then is employed to transduce a packaging cell line to form a producer cell line. Examples of packaging cells which may be transfected include, but are not limited to the PE501, PA317, ψ2, ψ-AM, PA12, T19-14×, VT-19-17-H2, ψCRE, ψCRIP, GP+E-86, GP+envAM12, and DAN cell lines. The vector containing the CA125 promoter and the polynucleotide encoding a polypeptide that is capable of providing for the inhibition, prevention, or destruction of the growth of the tumor cells upon expression of the polynucleotide may transduce the packaging cells through any means known in the art. Such means include, but are not limited to, electroporation, the use of liposomes, and CaPO₄ precipitation.

The producer cells then are administered directly to or adjacent to the tumor in an amount effective to inhibit, prevent, or destroy the growth of the tumor upon subsequent radiation therapy. In general, the producer cells are administered in an amount tolerated by the patient, it is desirable to inject as many producer cells as possible. The exact amount of producer cells to be administered is dependent upon various factors, including but not limited to, the type of the tumor and the size of the tumor.

In general, the producer cells are administered directly to or adjacent to the tumor by injection. The producer cells are administered in combination with a pharmaceutically acceptable carrier suitable for administration to a patient. The carrier may be a liquid carrier such as, for example, a saline solution.

Upon administration of the producer cells to the tumor, the producer cells generate viral particles. The viral particles then transduce the surrounding tumor cells. Because tumor cells, and in particular cancerous tumor cells, in general are actively replicating cells, the retroviral particle would be integrated into and expressed preferentially or exclusively in the tumor cells as opposed to normal cells.

In accordance with an aspect of the invention, there is provided a method of treating an ovarian tumor in a human host. The method comprises transducing ovarian tumor cells in vivo or in vitro with a polynucleotide encoding a therapeutic gene that is under the control of a CA125/M17S2 promoter. This invention involves targeting a gene-of-interest to the diseased ovarian cancer site so that the protein encoded by the gene is expressed, thereby directly or indirectly ameliorating the diseased state.

After infecting a susceptible cell, the transgene driven by a specific promoter in the vector expresses the protein encoded by the gene. The use of the highly specific ovarian cancer cell-specific gene vector will allow selective expression of the specific genes in ovarian cancer cells.

The present invention relates to a process for administering modified vectors into ovarian cancer cells to treat ovarian cancer or disorders associated therewith. More particularly, the invention relates to the use of vectors carrying functional therapeutic genes to produce molecules that are capable of directly or indirectly affecting ovarian cancer cells to repair damage sustained by the cells from defects, disease or trauma.

Preferably, for treating defects, disease or damage of ovarian cancer cells, vectors of the invention include a therapeutic gene or transgenes, for example a gene encoding TK. The genetically modified vectors are administered into the ovarian cancer cells to treat defects, disease such as ovarian cancer by introducing a therapeutic gene product or products into the ovarian cancer cells that enhance the production of endogenous molecules that have ameliorative effects in vivo.

The basic tasks in the present method of the invention are isolating the gene of interest, selecting the proper vector vehicle to deliver the gene of interest to the body, administering the vector having the gene of interest into the body, and achieving appropriate expression of the gene of interest. The present invention provides packaging the cloned genes, i.e. the genes of interest, in such a way that they can be injected directly into the bloodstream or relevant organs of patients who need them. The packaging will protect the foreign DNA from elimination by the immune system and direct it to appropriate tissues or cells.

Along with the human or animal gene of interest another gene, e.g., a selectable marker, or reporter gene, can be inserted that will allow easy identification of cells that have incorporated the modified retrovirus. The critical focus on the process of gene therapy is that the new gene must be expressed in target cells at an appropriate level with a satisfactory duration of expression.

The methods described below for modifying vectors, and administering such modified vectors into ovarian cancer cells are merely for purposes of illustration and are typical of those that might be used. However, other procedures may also be employed, as is understood in the art.

Most of the techniques used to construct vectors and the like are widely practiced in the art, and most practitioners are familiar with the standard resource materials which describe specific conditions and procedures. However, for convenience, the following paragraphs may serve as a guideline.

Construction of suitable vectors containing the desired therapeutic gene coding and control sequences employs standard ligation and restriction techniques, which are well understood in the art (see, e.g., MOLECULAR CLONING: A LABORATORY MANUAL, 3^{rd} ed., Sambrook et al., Cold Spring Harbor Laboratory Press, Cold Spring Harbor, N.Y. (2001)). Isolated plasmids, DNA sequences, or synthesized oligonucleotides are cleaved, tailored, and religated in the form desired.

The DNA sequences which form the vectors are available from a number of sources. Backbone vectors and control systems are generally found on available “host” vectors which are used for the bulk of the sequences in construction. Typical sequences have been set forth above. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleoside derivatives. The entire sequence for genes or cDNA's of sizable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence. See, e.g., Edge, Nature 292:756 (1981); Nambair et al., Science 223:1299 (1984); Jay, J. Biol. Chem. 259:6311 (1984).

Synthetic oligonucleotides are prepared by either the phosphotriester method as described by Edge et al., Nature (supra) and Duckworth et al., Nucleic Acids Res. 9:1691 (1981) or the phosphoramidite method as described by Beaucage et al., Tet. Letts. 22:1859 (1981) and Matteucci et al., J. Am. Chem. Soc. 103:3185 (1981) and can be prepared using commercially available automated oligonucleotide synthesizers. Kinasing of single strands prior to annealing or for labeling is achieved using an excess, e.g., approximately 10 units of polynucleotide kinase to 1 nmole substrate in the presence of 50 mM Tris, pH 7.6, 10 mM MgCl₂, 5 mM dithiothreitol, 1-2 mM ATP, 1.7 pmoles γ32P-ATP (2.9 mCi/mmole), 0.1 mM spermidine, 0.1 mM EDTA.

Site-specific DNA cleavage is performed by treating with the suitable restriction enzyme (or enzymes) under conditions which are generally understood in the art, and the particulars of which are specified by the manufacturer of these commercially available restriction enzymes (see, e.g., New England Biolabs Product Catalog). In general, about 1 μg of plasmid or DNA sequences is cleaved by one unit of enzyme in about 20 μl of buffer solution. Typically, an excess of restriction enzyme is used to insure complete digestion of the DNA substrate. Incubation times of about one hour to two hours at about 37° C. are workable, although variations can be tolerated. After each incubation, protein is removed by extraction with phenol/chloroform, and may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in METHODS IN ENZYMOLOGY, NUCLEIC ACIDS, PART I (vol. 65), Grossman et al., Academic Press (1980).

Restriction cleaved fragments may be blunt ended by treating with the large fragment of E. coli DNA polymerase I (Klenow) in the presence of the four deoxynucleotide triphosphates (dNTPs) using incubation times of about 15 to 25 minutes at 20° C. to 25° C. in 50 mM Tris (pH 7.6) 50 mM NaCl, 6 mM MgCl₂, 6 mM DTT and 5-10 μM dNTPs. The Klenow fragment fills in at 5′ sticky ends but chews back protruding 3′ single strands, even though the four dNTPs are present. If desired, selective repair can be performed by supplying only one of the dNTPs, or with selected dNTPs, within the limitations dictated by the nature of the sticky ends. After treatment with Klenow, the mixture is extracted with phenol/chloroform and ethanol precipitated. Treatment under appropriate conditions with S1 nuclease or Bal-31 results in hydrolysis of any single-stranded portion.

Ligations are performed in 10-50 μl volumes under the following standard conditions and temperatures using T4 DNA ligase. Ligation protocols are standard (see, e.g., METHODS IN ENZYMOLOGY, GENE EXPRESSION TECHNOLOGY (vol. 185) Goeddel, Academic Press (1990)).

In vector construction employing “vector fragments,” the vector fragment is commonly treated with bacterial alkaline phosphatase (BAP) or calf intestinal alkaline phosphatase (CIP) in order to remove the 5′ phosphate and prevent religation of the vector. Alternatively, religation can be prevented in vectors which have been double digested by additional restriction enzyme digestion of the unwanted fragments.

Once the components of the desired vectors are thus available, they can be excised and ligated using standard restriction and ligation procedures.

Suitable vectors include viral vector systems, e.g., ADV, RV, and AAV (METHODS IN ENZYMOLOGY, GENE EXPRESSION TECHNOLOGY (vol. 185), Goeddel, Academic Press (1990)).

Many methods for inserting functional DNA transgenes into cells are known in the art. For example, non-vector methods include nonviral physical transfection of DNA into cells; for example, microinjection (DePamphilis et al., BioTechnique 6:662-80 (1988)); liposomal mediated transfection (Felgner et al., Proc. Natl. Acad. Sci. USA 84:7413-17 (1987), Feigner et al., Focus 11:21-5 (1989), and Feigner et al., Proc. West. Pharmacol. Soc. 32:115-21 (1989)) and other methods known in the art.

One way to get DNA into a target cell is to put it inside a membrane bound sac or vesicle such as a spheroplast or liposome, or by calcium phosphate precipitation (CaPO₄) (Graham et al., Virology 52:456 (1973); Schaefer-Ridder et al., Science 215:166 (1982); Stavridis et al., Exp. Cell Res. 164:568-72 (1986)).

A vesicle can be constructed in such a way that its membrane will fuse with the outer membrane of a target cell. The vector of the invention in vesicles can home into the ovarian cancer cells. The spheroplasts are maintained in high ionic strength buffer until they can be fused through the mammalian target cell using fusogens such as polyethylene glycol. Liposomes are artificial phospholipid vesicles. Vesicles range in size from 0.2 to 4.0 μm and can entrap 10% to 40% of an aqueous buffer containing macromolecules. The liposomes protect the DNA from nucleases and facilitate its introduction into target cells. Transfection can also occur through electroporation.

Before administration, the modified vectors are suspended in complete PBS at a selected density for injection. In addition to PBS, any osmotically balanced solution which is physiologically compatible with the subject may be used to suspend and inject the modified vectors into the host.

For injection, the cell suspension is drawn up into the syringe and administered to anesthetized recipients. Multiple injections may be made using this procedure. The viral suspension procedure thus permits administration of genetically modified vectors to any predetermined site in the ovarian tumors, is relatively non-traumatic, allows multiple administrations simultaneously in several different sites or the same site using the same viral suspension. Multiple injections may consist of a mixture of therapeutic genes.

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many prokaryotic genes consists of the promoter and in some cases enhancer or regulator elements (Banerji et al., Cell 27:299 (1981); Corden et al., Science 209:1406 (1980); and Breathnach et al., Ann. Rev. Biochem. 50:349 (1981)).

For retroviruses, control elements involved in the replication of the retroviral genome reside in the long terminal repeat (LTR) (THE MOLECULAR BIOLOGY OF TUMOR VIRUSES: RNA TUMOR VIRUSES, Weiss et al., eds., Cold Spring Harbor Laboratory, Cold Spring Harbor, N.Y. (1982)). Moloney murine leukemia virus (MLV) and Rous sarcoma virus (RSV) LTRs contain promoter and enhancer sequences (Jolly et al., Nucleic Acids Res. 11:1855 (1983); ENHANCERS AND EUKARYOTIC GENE EXPRESSION, Gluzman et al., eds., Cold Spring Harbor Laboratories, Cold Spring Harbor, N.Y. (1983)).

Promoter and enhancer regions of a number of non-viral promoters have also been described (Schmidt et al., Nature 314:285 (1985); Rossi et al., Proc. Natl. Acad. Sci. USA 84:5590-4 (1987)).

The present invention provides methods for maintaining and increasing expression of therapeutic genes using the tissue-specific CA125/M17S2 promoter.

In addition to using viral and non-viral promoters to drive therapeutic gene expression, an enhancer sequence may be used to increase the level of therapeutic gene expression. Enhancers can increase the transcriptional activity not only of their native gene but also of some foreign genes (Armelor, Proc. Natl. Acad. Sci. USA 70:2702 (1973)).

For example, in the present invention, CMV enhancer sequences can be used with the CA125/M17S2 promoter to increase therapeutic gene expression. Therapeutic gene expression may also be increased for long term stable expression after injection using cytokines to modulate promoter activity.

The methods of the invention are exemplified by preferred embodiments in which modified vectors carrying a therapeutic gene are injected into a subject.

In a first embodiment, a protein product is expressed comprising growing the host vector system of the invention so as to produce the protein in the host and recovering the protein so produced. This method permits the expression of genes of interest in both unicellular and multicellular organisms. For example, in an in vitro assay, ovarian cancer cells having the vector of the invention comprising a gene of interest (e.g., the ras gene) may be used in microtiter wells as an unlimited assay for the ras gene product. A sample from a subject would be added to the wells to detect the presence of antibodies directed against the ras gene. This assay can aid in the quantitative and qualitative determination of the presence of ras antibodies in the sample for the clinical assessment of whether the subject's immune system is combating the disease associated with elevated levels of ras.

In a second embodiment ovarian cancer is treated via gene therapy, i.e., the correction of a disease phenotype in vivo through the use of the nucleic acid molecules of the invention.

In accordance with the practice of this invention, the subject of the gene therapy may be a human, equine, porcine, bovine, murine, canine, feline, or avian subject. Other warm blooded animals are also included in this invention.

The most effective mode of administration and dosage regimen for the molecules of the present invention depends upon the exact location of the ovarian tumor being treated, the severity and course of the cancer, the subject's health and response to treatment and the judgment of the treating physician. Accordingly, the dosages of the molecules should be titrated to the individual subject. The molecules may be delivered directly or indirectly via another cell, autologous cells are preferred, but heterologous cells are encompassed within the scope of the invention.

The interrelationship of dosages for animals of various sizes and species and humans based on mg/m² of surface area is described by Freireich et al., Cancer Chemother Rep. 50(4):219-44 (1966). Adjustments in the dosage regimen may be made to optimize the tumor cell growth inhibiting and killing response, e.g., doses may be divided and administered on a daily basis or the dose reduced proportionally depending upon the situation (e.g., several divided dose may be administered daily or proportionally reduced depending on the specific therapeutic situation).

It would be clear that the dose of the molecules of the invention required to achieve cures may be further reduced with schedule optimization.

Since the CA125/M17S2 promoter of the invention exhibits tissue specificity, it can only be activated in the targeted tissue, i.e., ovarian cancer cells. Therefore, the genes of interest driven by the CA125/M17S2 promoter will be differentially expressed in these cells, minimizing systemic toxicity.

The specificity of the CA125/M17S2 promoter and regulatory regions of the invention may be exploited in any of a number of recombinant nucleotide protocols to direct expression to specific cells and tissues. The CA125/M17S2 gene is expressed in ovarian cancer cells and thus its promoter will cause expression of operably linked sequences for these cells and other cells and tissues. The temporal and spatial specificity of this promoter can be used when operably linked to other non-naturally occurring nucleotide sequences to target expression of these alternate genes in a similar manner. Methods to identify promoters from other mammalian species for CA125/M17S2 include techniques known in the art as well as provided herein.

In one aspect, the invention provides an isolated promoter polynucleotide from a CA125/M17S2 gene. preferably, the polynucleotide comprises a promoter region from the CA125/M17S2 gene comprising a sequence set forth in SEQ ID NO: 1 and in FIG. 1, or a variant thereof.

In a further aspect, the invention provides isolated promoter and nucleic acid molecules from the CA125/M17S2 gene including, for example, polynucleotides derived from such molecules as unprocessed RNAs, ribosome RNAs, mRNAs, cDNAs, genomic DNAs, B- and Z-DNAs. Further embodiments of the invention include biologically, diagnostically, prophylactically, clinically, or therapeutically useful polynucleotides and variants thereof and compositions comprising the same.

Another aspect of the invention relates to isolated polynucleotides including, for example, polynucleotides closely related to a CA125/M17S2 promoter having a polynucleotide sequence set forth in SEQ ID NO: 1 and FIG. 1, and variants thereof.

Using the information provided herein such as the promoter polynucleotide sequence of set forth in SEQ ID NO: 1 and FIG. 1, other promoter polynucleotides of the invention may be obtained using standard cloning and screening methods such as those for cloning and sequencing chromosomal (genomic) DNA fragments as disclosed herein. For example, to obtain a polynucleotide sequence of the invention such as the polynucleotide sequence set forth in SEQ ID NO: 1 and FIG. 1, typically a library of clones of chromosomal DNA of a mammalian species or some other suitable host is probed with a radiolabeled oligonucleotide preferably a 7-mer or longer derived from a partial sequence. Clones carrying DNA identical to that of the probe can then be distinguished using stringent hybridization conditions. By sequencing the individual clones as identified by the hybridization with sequencing primers designed from the original polynucleotide sequence, it is possible to extend the polynucleotide sequence in both directions to determine a functional promoter region sequence or full-length gene sequence. Conveniently such sequencing is performed, for example, using denatured double-stranded DNA prepared from a plasmid clone. Suitable techniques for accomplishing this objective is described in MOLECULAR CLONING: A LABORATORY MANUAL, 3^{rd} ed., Sambrook et al., Cold Spring Harbor Laboratory Press, Cold Spring Harbor, N.Y. (2001). Direct genomic DNA sequencing may also be performed to obtain a promoter sequence of expressively linked full-length sequence.

Another very important method that can be used to identify cell type specific promoters that allow identification of genes expressed in a single cell is enhancer detection (O'Kane et al., Proc. Natl. Acad. Sci. USA 84:9123-7 (1987)). This method was first developed in Drosophila and rapidly adapted to mice and plants (Wilson et al., Genes Dev., 3:1301-13 (1989); Skarnes, Biotechnology 8:827-31 (1990); Topping et al., Development 112:1009-19 (1991); Sundaresan et al., Genes Dev., 9:1797-1810 (1995)).

In a further aspect, the present invention provides for an isolated polynucleotide comprising or consisting of a polynucleotide sequence which has at least 70% identity, preferably at least 80% identity, more preferably at least 90% identity, yet more preferably at least 95% identity, even more preferably at least 97-99%, or exact identity to the polynucleotide sequence set forth in SEQ ID NO: 1 and FIG. 1 over the entire length of the sequence ID, or polynucleotides which hybridize under conditions of high stringency thereto and yet retain promoter activity as determined by the assays herein.

Promoter polynucleotides from a polynucleotide encoding a CA125/M17S2 polypeptide, including homologues and orthologues from species other than human, may be obtained by a process which comprises screening an appropriate library under stringent hybridization conditions with a labeled detectable probe consisting of or comprising of sequences of SEQ ID NO: 1, or a fragment thereof, and isolating the promoter and/or full length gene and/or genomic clones containing the polynucleotide sequence, as previously described.

Preferred embodiments are polynucleotides that retain substantially the same biological function or activity as the promoter region of SEQ ID NO: 1. The promoter polynucleotides of the invention may be employed for example as research reagents and materials for discovery and treatment of, and diagnostics for diseases.

Assays of the invention may be performed by determining the effect of transcript level on cell phenotype. These assays will help to characterize, among other things, temporal relevance of transcription to phenotype. promoter polynucleotides of the invention may be used for over-production of heterologous proteins in eukaryotes and prokaryotes. promoter polynucleotides of the invention may also be used to assess the binding of small molecules, substrates and ligands in, for example, cells, cell free preparations, chemical libraries, and natural product mixtures to identify agonists and antagonists of promoter activity. These substrates and ligands may be natural substrates and ligands, or may be structural or functional mimetics.

Accordingly, in a further aspect, the present invention provides for a method of screening compounds to identify those which stimulate or which inhibit the function of the promoter polynucleotide of the invention as well as related polynucleotides.

Compounds may be identified from a variety of sources, for example cells, cell free preparations, chemical libraries and natural product mixtures. Such agonists, antagonists, or inhibitors so identified may be natural or modified substrates, ligands, receptors, enzymes, etc., or may be structural or functional mamatics thereof. These screening methods may simply measure the binding of a candidate compound to the promoter polynucleotide or to cells or membranes bearing the promoter polynucleotide. Alternatively the screening method may involve competition with a labeled competitor. Further, these screening methods may test whether the candidate compound results in a signal generated by activation or inhibition of the promoter polynucleotide using detection systems appropriate to the cells comprising the promoter polynucleotide. Inhibitors of activation are generally assayed in the presence of a known agonist and the effect on activation by the agonist by the presence of the candidate compound is observed. Constitutively active promoter polynucleotides and/or constitutively expressed polynucleotides may be employed in screening methods for inverse agonists or inhibitors in the absence of an agonist or inhibitor by testing whether the candidate compound results in inhibition of activation of the polynucleotide, as the case may be.

Further, the screening methods may simply comprise the steps of mixing a candidate compound with a solution containing a promoter polynucleotide of the invention to form a mixture measuring CA125/M17S2 promoter polynucleotide activity in the mixture, and comparing the CA125/M17S2 promoter polynucleotide activity of the mixture to a standard.

The methods of screening may involve high throughput techniques. For example to screen for agonists or antagonists, a synthetic reaction mix, a cellular compartment, such as a membrane, cell envelope or cell wall, or a preparation of any thereof, comprising a CA125/M17S2 promoter polynucleotide and a labeled substrate or ligand of such polynucleotide is incubated in the absence or presence of a candidate molecule that may be a CA125/M17S2 promoter agonist or antagonist. The ability of the candidate molecule to agonize or antagonize the CA125/M17S2 promoter polynucleotide is reflected in decreased binding of the labeled ligand or decreased production of the product from such substrate. Molecules that bind gratuitously (i.e., without inducing the effects of CA125/M17S2 promoter polynucleotide) are most likely to be good antagonists. Molecules that bind well and, as the case may be, increase the rate of product production from substrate, increase signal transaction or increase chemical channel activity are agonists. Detection of the rate or level as the case may be production of the product from the substrate signal transaction or chemical channel activity may be enhanced by using reporter system. Reporter systems that may be useful in this regard include but are not limited to colormetric, labeled substrate converted into product, a reporter gene that is responsive to changes in CA125/M17S2 promoter polynucleotide activity and binding assays known in the art.

Promoter polynucleotides of the invention may be used to identify promoter binding proteins, such as sigma factors, if any, for such polynucleotide, through standard binding techniques known in the art, for example, gel retardation assays. Other of these techniques include, but are not limited to, ligand binding and crosslinking assays in which the promoter polynucleotide is labeled with a radioactive isotope (for instance, ³²P), chemically modified (for instance, biotinylated or fluorescent tagged), or fused to a polynucleotide sequence suitable for detection or purification, and incubated with a source of the putative binding compound or ligand (e.g., cells, cell membranes, cell supernatants, tissue extracts, bodily materials). Other methods include biophysical techniques such as surface plasmon resonance and spectroscopy. These screening methods may also be used to identify agonists and antagonists of the promoter polynucleotide which compete with the binding of the polynucleotide to its ligand(s), if any. Standard methods for conducting such assays are well understood in the art.

The fluorescence polarization value for a fluorescently-tagged molecule depends on the rotational correlation time or tumbling rate. Protein-polynucleotide complexes, such as formed by CA125/M17S2 promoter polynucleotide associating with polypeptide or other factor, labeled to comprise a fluorescently-labeled molecule will have higher polarization values than a fluorescently labeled monomeric polynucleotide. It is preferred that this method be used to characterize small molecules that disrupt polypeptide-polynucleotide complexes.

Fluorescence energy transfer may also be used to characterize small molecules that interfere with the formation of CA125/M17S2 promoter polynucleotide-polypeptide dimers, trimers, tetramers or higher order structures, or structures formed by CA125/M17S2 promoter polynucleotide and a polypeptide or polypeptides. CA125/M17S2 promoter polynucleotide can be labeled with both a donor and acceptor fluorophore. Upon mixing of the two labeled species and excitation of the donor fluorophore, fluorescence energy transfer can be detected by observing fluorescence of the acceptor. Compounds that block dimerization will inhibit fluorescence energy transfer.

In other embodiments of the invention there are provided methods for identifying compounds which bind to or otherwise inject with and inhibit or activate an activity or expression of the promoter polynucleotide of the invention comprising: contacting a promoter polynucleotide of the invention with a compound to be screened under conditions to permit binding to or other interaction between the compound and the promoter polynucleotide to assess the binding to or other iron with the compound, such binding or interaction preferably being associated with a second component capable of providing a detectable signal in response to the binding or interaction of the promoter polynucleotide with the compound; and determining whether the compound binds to or otherwise interacts with and activates or inhibits an activity or expression of the promoter polynucleotide by detecting the presence or absence of a signal generated from the binding or interaction of the compound with the promoter polynucleotide.

Another example of an assay for CA125/M17S2 promoter agonists or antagonists is a competitive assay that combines CA125/M17S2 promoter and a potential agonist or antagonist with CA125/M17S2 promoter-binding molecules, recombinant CA125/M17S2 promoter binding molecules, natural substrates or ligands or substrate or ligand mimetics, under appropriate conditions for a competitive inhibition assay. CA125/M17S2 promoters can be labeled, such as by radioactivity or a calorimetric compound, such that the number of CA125/M17S2 promoter molecules bound to a binding molecule or converted to product can be determined accurately to assess the effectiveness of the potential antagonist or agonist.

Potential antagonists include, among others, small organic molecules, peptides, polypeptides that bind to a promoter polynucleotide of the invention and thereby inhibit or extinguish its activity or expression. potential antagonists also may be small organic molecules, a peptide, a polypeptide such as a closely related protein that binds the same sites on a binding molecule, such as a binding molecule, without inducing CA125/M17S2 promoter-induced activities, thereby preventing the action of CA125/M17S2 promoter polynucleotides by excluding CA125/M17S2 promoter polynucleotides from binding.

Potential antagonists include a small molecule that binds to and occupies the binding site of the promoter polynucleotide thereby preventing binding to cellular binding molecules, such that normal biological activity is prevented. Examples of small molecules include but are not limited to small organic molecules, peptides or peptide-like molecules. Other potential antagonists include antisense molecules (see Okano et al., J. Neurochem. 56:560-7 (1991); and ANTISENSE OLIGODEOXYNUCLEOTIDES AND ANTISENSE RNA: NOVEL PHARMACOLOGICAL AND THERAPEUTIC AGENTS, Weiss et al., ed., CRC Press, Boca Raton, Fla. (1987), for a description of these molecules).

Other examples of polypeptide antagonists include oligonucleotides or proteins which are closely related to the ligands, substrates, receptors, enzymes, etc., as the case may be, of the polynucleotide, e.g., a fragment of the ligands, substrates, receptors, enzymes, etc.; or small molecules which bind to the polynucleotide of the present invention but do not elicit a response, so that the activity of the polynucleotide is prevented.

Certain of the polynucleotides of the invention are biomimetics, functional mimetics of the natural CA125/M17S2 polynucleotide. These functional mimetics may be used for, among other things, antagonizing the activity of CA125/M17S2 promoter. Functional mimetics of the polynucleotides of the invention include but are not limited to truncated polynucleotides. For example, preferred functional mimetics include, a polynucleotide comprising the polynucleotide sequence set forth in SEQ ID NO: 1 lacking 5, 10, 20, 30, 40, 50, 60, 70 or 80 5′ and/or 3′ nucleotide residues, including fusion promoters comprising one or more of these truncated sequences. polynucleotides of these functional mimetics may be used to drive the expression of expression cassettes and marker genes. It is preferred that these cassettes comprise 5′ and 3′ restriction sites to allow for a convenient means to ligate the cassettes together when desired. It is further preferred that these cassettes comprise gene expression signals known in the art or described elsewhere herein.

It will be readily appreciated by the skilled artisan that a polynucleotide of the present invention may also be used in a method for the structure-based design of an agonist, antagonist or inhibitor of the promoter polynucleotide, by: (a) determining in the first instance the three-dimensional structure of the polynucleotide, or complexes thereof, (b) deducing the three-dimensional structure for the likely reactive site(s), binding site(s) or motif(s) of an agonist, antagonist or inhibitor; (c) synthesizing candidate compounds that are predicted to bind to or react with the deduced binding site(s), reactive site(s), and/or motif(s); and (d) testing whether the candidate compounds are indeed agonists, antagonists or inhibitors.

It will be further appreciated that this will normally be an iterative process, and this iterative process may be performed using automated and computer-controlled steps.

The following description sets forth the general procedures involved in practicing the present invention. To the extent that specific materials are mentioned, it is merely for purposes of illustration and is not intended to limit the invention. Unless otherwise specified, general cloning procedures are utilized, such as those set forth in MOLECULAR CLONING: A LABORATORY MANUAL, 3^{rd} ed., Sambrook et al., Cold Spring Harbor Laboratory Press, Cold Spring Harbor, N.Y. (2001) or CURRENT PROTOCOLS IN MOLECULAR BIOLOGY, Ausubel et al., John Wiley & Sons (1999).

Hosts and Control Sequences

Both prokaryotic and eukaryotic systems may be used to express the nucleotide constructs of the invention; prokaryotic hosts are, of course, the most convenient for cloning procedures. prokaryotes most frequently are represented by various strains of E. coli; however, other microbial strains may also be used. plasmid vectors which contain replication sites, selectable markers and control sequences derived from a species compatible with the host are often used in addition to the promoter of the invention; for example, E. coli is typically transformed using derivatives of pBR322, a plasmid derived from an E. coli species by Bolivar et al., Gene 2:95 (1977). pBR322 contains genes for ampicillin and tetracycline resistance, and thus provides multiple selectable markers which can be either retained or destroyed in constructing the desired vector. Commonly used prokaryotic control sequences which are defined herein to include promoters for transcription initiation, optionally with an operator, along with ribosome binding site sequences, include such commonly used promoters as the beta-lactase (penicillinase) and lactose (lac) promoter systems (Chang et al., Nature 198:1056 (1977)) and the tryptophan (trp) promoter system (Goeddel et al., Nucleic Acids Res. 8:4057 (1980)) and the lambda derived P_{L} promoter and N-gene ribosome binding site (Shimatake et al., Nature 292:128 (1981)).

In addition to bacteria, eukaryotic microbes, such as yeast, may also be used as hosts. Laboratory strains of Saccharomyces cerevisiae, Baker's yeast, are most used although a number of other strains or species are commonly available. Vectors employing, for example, the 2μ origin of replication of Broach, Methods Enzymol. 101:307 (1983), or other yeast compatible origins of replication (see, e.g., Stinchcomb et al., Nature 282:39 (1979), Tschumper et al., Gene 10:157 (1980) and Clarke et al., Methods Enzymol. 101:300 (1983)) may be used. Control sequences for yeast vectors include promoters for the synthesis of glycolytic enzymes (Hess et al., J. Adv. Enzyme Reg. 7:149 (1968); Holland et al., Biochemistry 17:4900 (1978)). Additional promoters known in the art include the promoter for 3-phosphoglycerate kinase (Hitzeman et al., J. Biol. Chem. 255:2073 (1980)). Other promoters, which have the additional advantage of transcription controlled by growth conditions and/or genetic background are the promoter regions for alcohol dehydrogenase 2, isocytochrome C, acid phosphatase, degradative enzymes associated with nitrogen metabolism, the alpha factor system and enzymes responsible for maltose and galactose utilization. It is also believed terminator sequences are desirable at the 3′ end of the coding sequences. Such terminators are found in the 3′ untranslated region following the coding sequences in yeast-derived genes.

It is also, of course, possible to express genes encoding polypeptides in eukaryotic host cell cultures derived from multicellular organisms. See, e.g., U.S. Pat. No. 4,399,216. These systems have the additional advantage of the ability to splice out introns and thus can be used directly to express genomic fragments. Useful host cell lines include bovine epithelial cells, VERO and HeLa cells, and Chinese hamster ovary (CHO) cells. Expression vectors for such cells ordinarily include promoters and control sequences compatible with mammalian cells such as the promoter of the invention in combination with, for example, the commonly used early and late promoters from Simian Virus 40 (SV 40) (Fiers et al., Nature 273:113 (1978)), or other viral promoters such as those derived from polyoma, Adenovirus 2, bovine papilloma virus, or avian sarcoma viruses. The controllable promoter, hMT1I (Karin et al., Nature 299:797-802 (1982)) may also be used. General aspects of mammalian cell host system transformations have been described in U.S. Pat. No. 4,399,216. It now appears, also that “enhancer” regions are important in optimizing expression; these are, generally, sequences found upstream or downstream of the promoter region in non-coding DNA regions. Origins of replication may be obtained, if needed, from viral sources. However, integration into the chromosome is a common mechanism for DNA replication in eukaryotes.

The invention also provides in vitro methods for assaying for the activity of the CA125/M17S2 promoters of the invention comprising introducing to an appropriate cell a nucleotide construct comprising a gene under the control of a CA125/M17S2 promoter operably linked thereto, and assaying for the expression of the gene, wherein expression of the gene indicates that the promoter is active in the cell, and wherein lack of expression of the gene indicates that the promoter is not active or has reduced activity in the cell. Preferably, such genes operably linked to the CA125/M17S2 promoter include a therapeutic gene or transgenes, for example a gene encoding TK.

In order that the invention described herein may be more fully understood, the following example is set forth. It should be understood that this example is for illustrative purposes only, and is not to be construed as limiting the scope of this invention in any manner.

### EXAMPLE 1

### Cloning and Analysis of CA125/M17S2 Promoter PCR Amplification and Cloning of CA125P1431

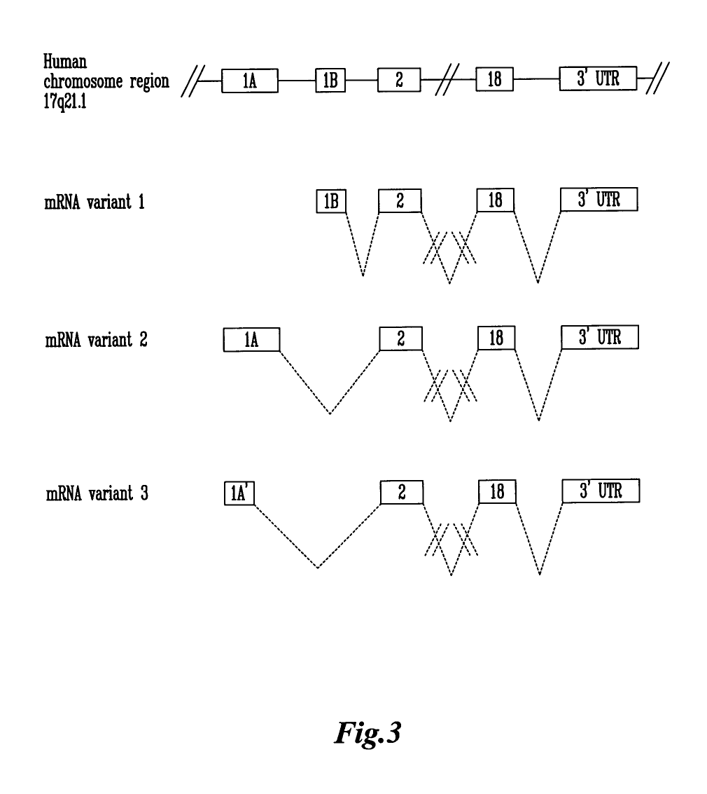

A 1431-bp promoter fragment of the CA125/M17S2 gene, CA125P1431, was amplified by Polymerase Chain Reaction (PCR) using designed primers, 5′-CCGTCCAGAACGTCTCAGCGAGCTCACGAC-3′ (SEQ ID NO:2) and 5′-CAAGGACCGTCCGCTACCGGCTCTGCCGCTATC-3′ (SEQ ID NO:3), based on the sequence of promoter region of human 1A1.3B sequence in GenBank (accession number U72483). The amplified fragment was cloned into vector pCR-XL-TOPO of TOP XL PCR Cloning Kit (Invitrogen) following manufacturer's instruction, and the cloned insert was confirmed by sequencing. The sequence of CA125p1431 is shown in FIG. 1, and is embodied in SEQ ID NO:1. Alternatively, spliced CA125/M17S2 mRNA (FIG. 3) is transcribed from either exon 1A or exon 1B. The CA125p1431 fragment contains exon 1A and partial sequence of exon 1B of the CA125/M17S2 gene.

Construction of pPL-EGFP

Plasmid vector pEGFP-N1 (Clontech) was digested with restriction enzymes Ase I and Nhe I (New England BioLabs) to remove the CMV IE promoter. The vector back bone (without CMV IE promoter) was then retrieved by elution of DNA fragment with Zymoclean Gel DNA Recovery Kit (Zymo Research) following separation by agarose gel electrophoresis. After filling in the ends with T4 DNA polymerase (New England BioLabs) in the presence of dNTPs (200 μM each of dATP, dCTP, dGTP and dTTP), the vector fragment was self-circularized by T4 DNA ligase (New England BioLabs) to generate a vector, pPL-EGFP, which contains a promoterless enhanced green fluorescence protein gene.

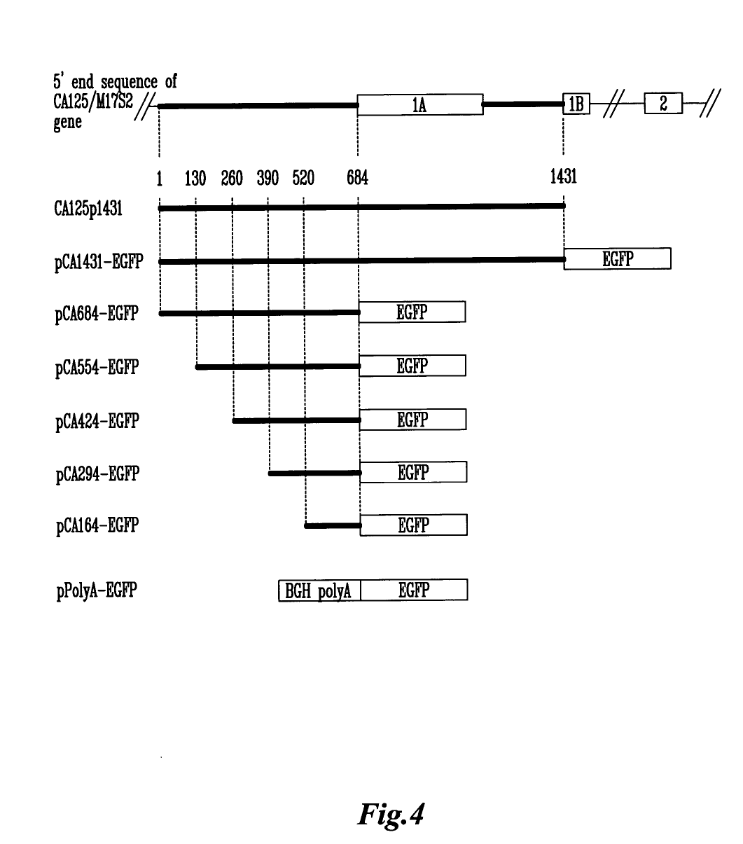

Construction of pCA125p1431-EGFP Plasmid pCA125p1431-EGFP, which contains the enhanced green fluorescence protein (EGFP) under the control of the cloned promoter, was constructed by inserting CA125p1431 fragment into the Sma I site of pPL-EGFP. The orientation of insert was confirmed by DNA sequencing. A series of deletions of the CA125p1431 fragment, CA684, CA554, CA424, CA294, and CA164, were amplified by PCRs with corresponding forward primers (each containing a Kpn I site) and reverse primers (each containing a BamH I site), respectively. These DNA fragments were individually cloned into the pPL-EGFP, using the Kpn I and BamH I restriction sites, to generate pCA684-EGFP, pCA554-EGFP, pCA424-EGFP, pCA294-EGFP, and pCA164-EGFP plasmid as shown in FIG. 4. A 300-bp DNA fragment containing the bovine growth hormone polyA signal (BGH polyA) was also amplified from plasmid pIRESneo (Clontech) by PCR for the construct of pPolyA-EGFP as a negative control plasmid in the analysis of the CA125/M17S2 promoter activity. The plasmids used for the promoter analysis were all examined by DNA sequencing.

Transfection of Plasmid DNAs into Ovarian Cancer Cell Lines

The HiSpeed Plasmid Midi Kit (Qiagen) was used to isolate plasmid DNAs for transfection experiments. The concentrations of isolated plasmid DNAs were determined by UV (260 nm) spectroscopy. To determine the optimal condition for DNA transfection into each of the cell lines (OVCAR4, IGROV, SK-OV-3, PA-1, SW626, HEK293, and DU145) in the analysis of CA125/M17S2 promoter, a six-well plate (1-3×10⁵ cells/well) was prepared for each cell line. Twenty-four hours after plate preparation, various amounts (1-2 μg) of pEGFP-N1 plasmid DNA and various volumes (2-6 μl) of FUGENE 6 Reagent (Roche) were used in the transfection experiment according to manufacturer's (Roche) instructions. Forty-eight hours after transfection setting, the cells were trypsinized and collected to determine the optimal transfection condition for each cell line by analyzing EGFP expression in transfected cells as determined by fluorescence activated cell sorting (FACS).

Analyses of Activities of CA125/M17S2 Promoter Deletions

Plasmid DNAs (pCA1431-EGFP, pCA684-EGFP, pCA554-EGFP, pCA424-EGFP, pCA294-EGFP, pCA164-EGFP, and pPolyA-EGFP), were individually transfected into each cell line using the optimal conditions determined in the transfection experiment. Promoter activities of the serial deletions of CA125p1431 fragment were determined as means fluorescence values by analyzing the EGFP expression in the transfected cells with FACS.

Results

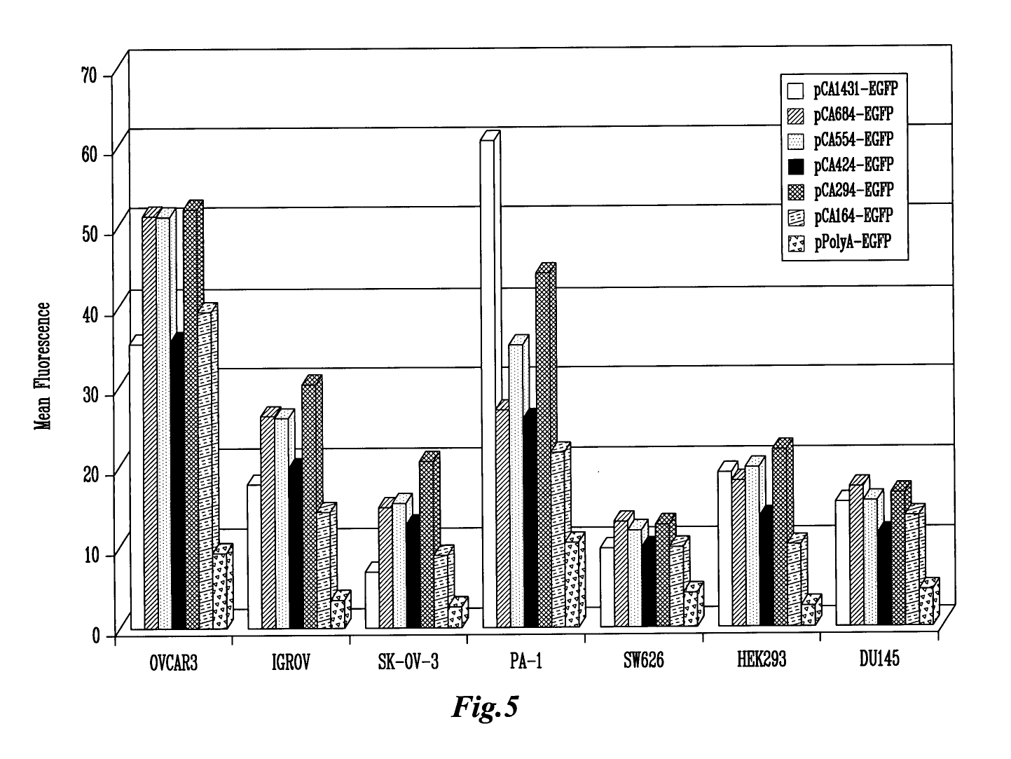

CA125/M17S2 promoter deletions showed much higher activities in OVCAR3 than in other ovarian cancer cell lines ((IGROV, SK-OV-3, and SWK626), one human embryonic kidney cell line (HEK293) and one human prostate cancer cell line (DU145)(FIG. 5). Lower EGFP expression from pCA424-EGFP than from pCA294-EGFP in each cell lines indicates that the nucleotide sequence located between nucleotide positions 390 and 521 of the CA125p1431 fragment (FIG. 1 and FIG. 4) contains cis-element(s) that down regulate expression under the control of CA125/M17S2 promoter. High EGFP expression from pCA1431-EGFP in PA-1 (a non-CA125-producing ovarian cancer cell line) suggests that CA125/M17S2 mRNA variant 1, which is expressed from exon 1B of CA125/M17S2 gene (FIG. 3), is preferentially transcribed in PA-1 cells. However, the predominant CA125/M17S2 mRNA isoform in OVCAR3 cells is either mRNA variant 2 or mRNA variant 3 (FIG. 3), as implied by higher EGFP expression from each of pCA-684-EGFP, pCA554-EGFP, pCA-424-EGFP, pCA294-EGFP, and pCA164-EGFP in OVCAR3 cells than in the other tested cells. These results were consistent with the observation of alternative transcriptional initiation and mRNA splicing of the CA125/M17S2 gene in different tissues and cell lines. These results also suggest that a CA125/M17S2 mRNA variant transcribed from exon 1A (mRNA variant 2 or 3) is highly expressed in CA125/M17S2-producing cancer cells.

### REFERENCES

1. Bast R. C., et al. (1981) Reactivity of a monoclonal antibody with human ovarian carcinoma. J. Clin. Invest., 68 (5): 1331-7.

2. Klug T. L., et al. (1984) Monoclonal antibody immunoradiometric assay for an antigenic determinant (CA 125) associated with human epithelial ovarian carcinomas. Cancer Res., 44 (3): 1048-53.

3. Bon G. G., et al. (1996) Serum tumor marker immunoassys in gynecologic oncology: establishment of reference values. Am. J. Obstet. Gynecol., 174 (1 pt. 1): 107-14.

4. Nastad K., et al. (1998) CA125-epitopes and molecular size. Int. J. Biol. Markers, 13 (4): 196-9.

5. Campbell I. G., et al. (1994) A novel gene encoding a B-box protein within the BRCA1 region at 17q21.1. Hum. Mol. Genet., 3 (4): 589-94.

6. Whitehouse C., et al. (2002) NBR1 interacts with fasciculation and elongation protein zeta-1 (FEZ1) and calcium and integrin binding protein (CIB) and shows developmentally restricted expression in the neural tube. Eur. J. Biochem., 269: 538-45.

7. Yin B. W. T., Loyd K. O. (2001) Molecular cloning of the CA125 ovarian cancer antigen. J. Biol. Chem., 276 (29): 27371-5.

8. O'Brien T. J., et al. (2001) The CA125 gene: an extracellular superstructure dominated by repeat sequences. Tumour Biol., 22 (6): 348-66.

9. Kutteh W. H., Miller D. S., Mathis J. M. (1996) Immunologic characterization of tumor markers in human ovarian cancer cell lines. J. Soc. Gynecol. Investig., 3 (4): 216-22.

10. Whitehouse C., Solomon E. (2003) Current status of the molecular characterization of the ovarian cancer antigen CA125 and implications for its use in clinical screening. Gynecol. Oncol., 88: S152-7.

11. Dimitrov S., Brennerova M., Forejt J. (2001) Expression profiles and intergenic structure of head-to-head oriented Brca! and Nbr1 genes. Gene, 262: 89-98.

What is claimed is:

1. A purified and isolated nucleic acid molecule capable of directing transcription of operably linked sequences consistent with CA125/M17S2 expression.

2. The isolated nucleic acid molecule of claim 1, wherein the isolated nucleic acid molecule comprises SEQ ID NO: 1.

3. An isolated nucleic acid molecule having promoter activity comprising a polynucleotide selected from the group consisting of:
(a) the polynucleotide of SEQ ID NO: 1;
(b) a polynucleotide having at least 90% identity to the polynucleotide of SEQ ID NO:1; and
(c) a polynucleotide having the SEQ ID NO 1 and extending 1000 nucleotides upstream into the genomic sequence of human chromosome 17 and containing additional transcriptional regulatory elements that control the gene expression of CA125/M17S2 gene in a positive or negative way.
(d) a polynucleotide complementary to a polynucleotide of (a) through (c).

4. The isolated nucleic acid molecule of claim 3, wherein the polynucleotide is the polynucleotide of SEQ ID NO: 1.

5. A nucleic acid construct having ovarian cancer cell specific promoter activity comprising a first polynucleotide operably linked to a second polynucleotide, wherein the first polynucleotide is selected from the group consisting of:
(a) the polynucleotide of SEQ ID NO: 1;
(b) a polynucleotide having at least 90% identity to the polynucleotide of SEQ ID NO:1; and
(c) a polynucleotide having the SEQ ID NO 1 and extending 1000 nucleotides upstream into the genomic sequence of human chromosome 17 and containing additional transcriptional regulatory elements that control the gene expression of CA125/M17S2 gene in a positive or negative way.
(d) a polynucleotide complementary to a polynucleotide of (a) through (c); and
a second polynucleotide sequence the expression of which is desired in a host cell.

6. The nucleic acid construct of claim 5, wherein the first polynucleotide is the polynucleotide of SEQ ID NO: 1.

7. The nucleic acid construct of claim 5, wherein the second polynucleotide is selected from the group consisting of a reporter gene, a viral gene and a therapeutic gene.

8. A vector comprising a nucleic acid construct, wherein the nucleic acid construct comprises a first polynucleotide operably linked to a second polynucleotide, wherein the first polynucleotide is selected from the group consisting of:
(a) the polynucleotide of SEQ ID NO: 1;
(b) a polynucleotide having at least 90% identity to the polynucleotide of SEQ ID NO:1; and
(c) a polynucleotide having the SEQ ID NO 1 and extending 1000 nucleotides upstream into the genomic sequence of human chromosome 17 and containing additional transcriptional regulatory elements that control the gene expression of CA125/M17S2 gene in a positive or negative way.
(d) a polynucleotide complementary to a polynucleotide of (a) through (c); and
the second polynucleotide is a sequence, the expression of which is desired in a host cell.

9. The vector of claim 8, wherein the first polynucleotide is the polynucleotide of SEQ ID NO: 1.

10. The vector of claim 8, wherein the second polynucleotide is selected from the group consisting of a reporter gene, a viral gene and a therapeutic gene.

11. The vector of claim 8, wherein the vector is a viral vector.

12. The vector of claim 11, wherein the viral vector is selected from the group consisting of a retroviral vector, an adenoviral vector, and an adeno-associated viral vector.

13. A host cell comprising a vector, wherein the vector comprises a nucleic acid construct having promoter activity, wherein the nucleic acid construct comprises a first polynucleotide operably linked to a second polynucleotide, wherein the first polynucleotide is selected from the group consisting of:
(a) the polynucleotide of SEQ ID NO: 1;
(b) a polynucleotide having at least 90% identity to the polynucleotide of SEQ ID NO: 1; and
(c) a polynucleotide having the SEQ ID NO 1 and extending 1000 nucleotides upstream into the genomic sequence of human chromosome 17 and containing additional transcriptional regulatory elements that control the gene expression of CA125/M17S2 gene in a positive or negative way.
(d) a polynucleotide complementary to a polynucleotide of (a) through (c); and
said second polynucleotide is a sequence the transcription of which is desired in a host cell.

14. The host cell of claim 13, wherein the first polynucleotide is the polynucleotide of SEQ ID NO:1.

15. The host cell of claim 13, wherein the second polynucleotide is selected from the group consisting of a reporter gene and a therapeutic gene.

16. The host cell of claim 13, wherein the vector is a viral vector.

17. The host cell of claim 16, wherein the viral vector is selected from the group consisting of a retroviral vector, a herpes simplex vector, an adenoviral vector, and an adeno-associated viral vector.

18. A isolated nucleic acid molecule having CA125/M17S2 promoter activity in ovarian cancer cells, wherein the nucleotide positions between 390 and 521 of SEQ ID NO: 1 contains a cis-element.

19. The isolated nucleic acid of claim 18, wherein the cis-element is capable of down regulating expression.

20. An isolated nucleic acid molecule capable of inducing transcription of operable linked sequences in an ovarian cancer cell comprising a polynucleotide having at least 90% identity to the polynucleotide of SEQ ID NO: 1, wherein the nucleotide positions between 390 and 521 of SEQ ID NO:1 contains a cis-element.

21. The isolated nucleic acid of claim 20, wherein the cis-element is capable of down regulating expression.

22. A method of providing a nucleotide sequence to treat ovarian cancer, the method comprising:
administering to a subject a nucleic acid construct comprising a therapeutic gene under the control of a CA125/M17S2 promoter, wherein the nucleotide sequence of said promoter is set forth in SEQ ID NO: 1, such that the construct is effective in treating ovarian cancer.

23. The method of claim 23, wherein the subject is human.


SEQUENCE LISTING (3 sequences)

SEQ ID NO: 1 - DNA, 1431 bp, Homo sapiens
ccgtccagaacgtctcagcgagctcacgacgcgcagtcacgtttttttccccccctctacattgcagatgtggctcccaatgttgacgttggccaggacctttgcaaacaagccaggccaaaaagtttcaatatttacactggctgctttaataagggcattgatcttatcctccgtaaaggtcacctcatagtcctgcagaatgagggcagagtagatgcaggcaagctgggagacggaggccatagcgcgggcgagtgtggggctggggctgccggacgcggtgctactcaccggatgaagtgagggtctcaccccaacgcggccttagcttcctcggaaggaccgaacaccttggcggcagccgaggaaaggggttccacagttttaatttatctgtaattcccacgctttactgttgccacggaaaccgctgagcaatagcctctcagaataggaaatcaagacacagtcagaggaagggcgggacagaaagagcctagcatctctcggggctctgggttggccacccagtcctcccctggtgacataaaaagaaagagacggaaaaggaagaattctacctgagttcgccgtaaagcgcccgccctctcgcctctacgcttccagttgcggcttattacgtcacagtaattgctgtaccaaggtcagaatcgccacctgaggcctgaatatcagcgtaagatagtgtccaaagcagtcttaagaagaggtcccattaccccactctttccgccctaatggaggtctccagtttaggtaaataaaaggattgttgggaggtggagggaaagaactactatttccaacatgcattgcggaacgaaaggccttggccacactgttccttggaaactgtagtcttatggagaggaacatccaataccaaagcgggcacaattctcacggaaatccagtggatagattggagacctccgcgggcttatacatgtcaacagtaatggattggagtgttgttatgttctcctatcttgagagcagagactaggccaaaaaaagatacctacaactcctaggaagactacgattcccatccagccccacgagtctcgggcaagtagtcctctaaggtcagtggcctgcggggacgcagtgggcgccgaatttgcctggggaaggggaaatccgctctggcccacatctgcgcactcctagttccgcccctcagcctcaatgtttgttattgttgttcgggttcaggttgcttctgccccgccccatcgacgcaatctccaccaatcaatggcgtggtcgttttgagggacaagtggtgagagccaatcatcttggcgaacactcggagaaacaggggactagttactgtctttatccgccatgttagattcaccccacagggatagcggcagagccggtagcggacggtccttg

SEQ ID NO: 2 - DNA, 28 bp, Artificial Sequence
  oligonucleotide primer derived from promoter region of human 1A1. 3B (GenBank accession number U72483)
ccgtccagaacgtctcagcgagctcacg

SEQ ID NO: 3 - DNA, 33 bp, Artificial Sequence
  oligonucleotide primer derived from promoter region of human 1A1. 3B (GenBank accession number U72483)
caaggaccgtccgctaccggctctgccgctatc